(19) United States
(12) Patent Application Publication
(10) Pub. No.: US 2018/0092752 A1
(43) Pub. Date: Apr. 5, 2018

(54) Spinal Fusion Implant with Reducible Graft Aperture

(71) Applicant: NuVasive, Inc., San Diego, CA (US)

(72) Inventor: Gregory M. Williams, San Diego, CA (US)

(73) Assignee: Rory Schermerhorn, San diego, CA (US)

(21) Appl. No.: 15/706,653

(22) Filed: Sep. 15, 2017

Related U.S. Application Data
(63) Continuation of application No. 14/457,108, filed on Aug. 11, 2014, now abandoned.
(60) Provisional application No. 61/864,159, filed on Aug. 9, 2013.

(51) Int. Cl. A61F 2/44; A61F 2/30

(52) U.S. Cl. CPC A61F 2/4455; A61F 2002/4475; A61F 2002/30772

(57) ABSTRACT

A spinal implant (e.g. interbody spacer) with one or more apertures for the containment of bone graft or bone graft substitute. The implant may be packed with graft material prior to or subsequent to implantation. The implant has an integrated mechanism to reduce the volume of the bone graft aperture thereby compressing or repositioning graft material.

### CROSS REFERENCE TO RELATED APPLICATIONS

This application is a continuation of U.S. patent application Ser. No. 14/457,108, filed Aug. 11, 2014 which claims the benefit of priority to U.S. Provisional Application No. 61/864,159, which was filed on Aug. 9, 2013, the contents of which are incorporated by reference in their entirety as if part of this application.

### BACKGROUND

This application relates to interbody spinal fusion implants and methods of use thereof.

### SUMMARY

A spinal implant (e.g. interbody spacer) with one or more apertures for the containment of bone graft or bone graft substitute. The implant may be packed with graft material prior to or subsequent to implantation. The implant has an integrated mechanism to reduce the volume of the bone graft aperture thereby compressing or repositioning graft material. This allows for desired graft compaction, continuity, and contact with surrounding tissues and may create space for additional graft material to be delivered.

The implant addresses needs for the biological fusion process: graft compaction, continuity, and contact with endplates, which can be hard to achieve with current spinal implants. The implant can also facilitate graft delivery and proper impaction of graft into the implant after positioning of the implant in situ. Thus, this technology addresses current issues related to inadequately filled implants or inadequate continuity and contact of the graft material with surrounding bone due to bone graft loss upon insertion, anatomical incongruities with the implant, or surgical manipulation of the spine.

### BRIEF DESCRIPTION OF THE DRAWINGS

FIG. 1 is a top view of a spinal fusion implant according to an exemplary embodiment;

FIG. 2 is a top view of a spinal fusion implant according to an alternative embodiment;

FIG. 3 is a top view of a spinal fusion implant according to a third embodiment; and

FIG. 4 is a top view of the spinal fusion implant of FIG. 3 after the graft apertures have been compacted.

### DETAILED DESCRIPTION

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary sill in the art having the benefit of this disclosure. The spinal fusion implant and related methods disclosed herein boasts a variety of inventive features and components that warrant patent protection, both individually and in combination.

In general, the spinal fusion implants described in this document include at least one graft aperture and a mechanism for reducing the size of the graft aperture after the implant has been placed in a desired position within an intervertebral disc space. The implant may include one or more pieces which deform or move relative to one another in order to achieve the desired reduction in bone graft aperture. The mechanism of reduction may include a moveable element which slides or ratchets inward to a fixed/locked position, a moveable piece which may alternately reduce and expand the graft aperture allowing graft material to be “tamped,” or an element which expands to occupy what was previously graft aperture volume.

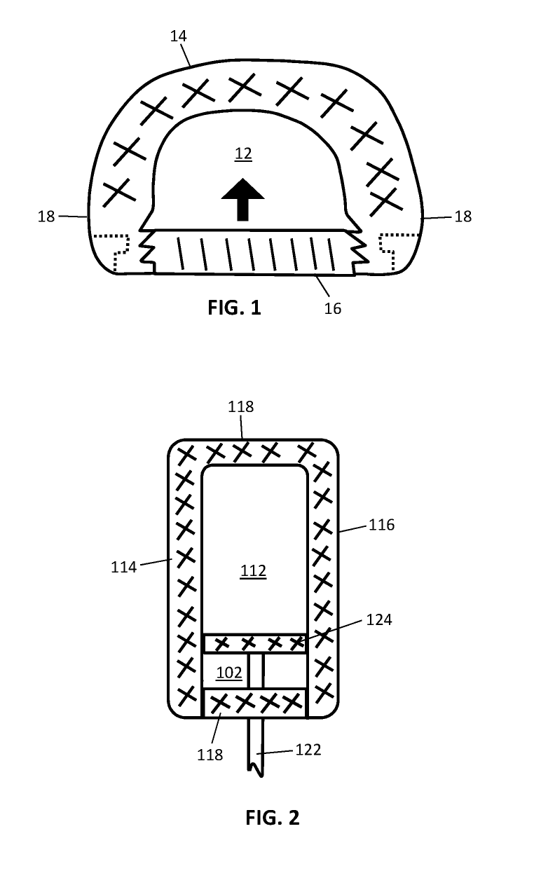

FIG. 1 illustrates an exemplary embodiment of the spinal fusion implant 10. The implant includes a graft aperture 12 defined by an anterior wall 14, a posterior wall 16 and a pair of opposing lateral walls 18. As illustrated in FIG. 1, the posterior wall 16 is moveable relative to the remaining three walls. However, it is appreciated that any one of the four walls can be the moveable wall. The moveable wall 16 has a mating feature 22 at each of its ends that engages a complementary mating feature 24 on the adjacent two walls to allow the moveable wall to be translated toward the opposing wall 14 thereby reducing the volume of the graft aperture 12. FIG. 1 illustrates a ratchet system as the mating features 22, 24.

FIG. 2 illustrates an alternative embodiment of the spinal fusion implant 110. According to this embodiment, the implant has a graft aperture 112 defined by an anterior wall 114, a posterior wall 116 and opposing lateral walls 118. According to this embodiment, the implant 110 includes an integrated tamp 120. The tamp 120 includes a block 122 and a handle 124 extending proximally from the block 122. The tamp 120 facilitates the compaction of bone graft within the aperture 112. Upon achieving satisfactory compaction of bone graft, the handle 124 is disengaged from the block 122. Optionally, the remaining void 102 between the block 122 and the proximal lateral wall 118 may be filled with additional graft after the tamp handle 124 is removed.

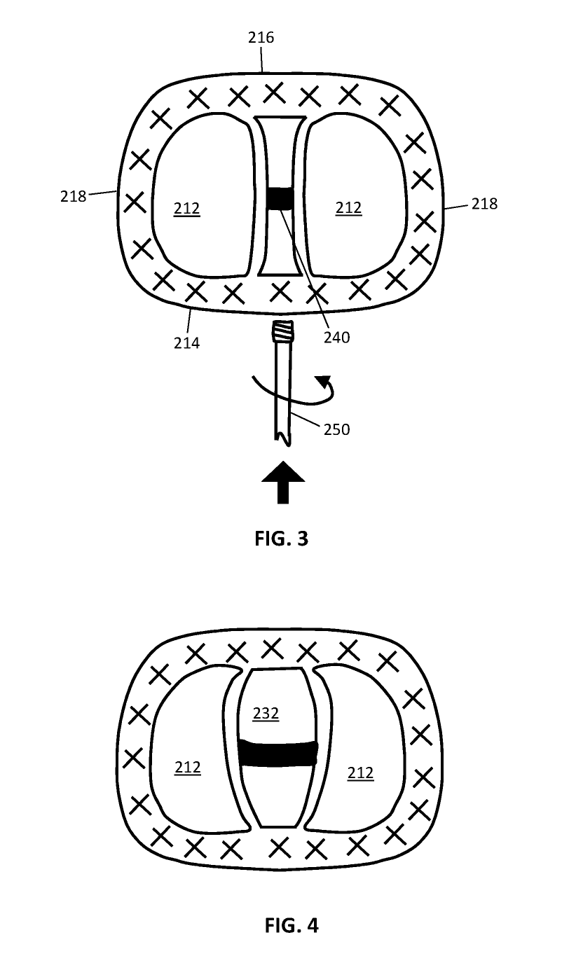

FIGS. 3-4 illustrate a third embodiment of the spinal fusion implant 210. According to this embodiment, the implant 210 has an anterior wall 214, a posterior wall 216 and opposing lateral walls 218 surrounding two graft apertures 212 and a central aperture 232. The central aperture 232 is bordered by flexible walls 226 and houses a rotating block 240. The implant 210 is inserted into the intervertebral disc space with the rotating block 240 in a first position as shown in FIG. 3. Upon placement of the implant 210 in a desired position within the disc space, an actuator tool 250 is engaged with the rotating block 240 and the block 240 is rotated 90° to a second position as shown in FIG. 4. In the second position, the rotating block urges the flexible walls 226 away from each other and toward the lateral walls 218, thereby reducing the volume of the two graft apertures 212.

In use, the implant in any of FIGS. 1-4 may be pre-packed with bone graft material prior to implantation by the surgical staff. The implant is then delivered through a retractor into the intervertebral space, vertebral body space, or similar bone defect where fusion is desired. If no graft material was pre-packed, the surgeon may deliver graft material into the aperture at this time. The surgeon may then utilize the integrated graft aperture reduction mechanism with associated instruments to achieve desired graft compaction or position.

A number of embodiments of the invention have been described. Nevertheless, it will be understood that various modifications may be made without departing from the spirit and scope of the invention. Accordingly, other embodiments are within the scope of the following claims.

What is claimed is:

1. A spinal fusion implant, comprising:
an anterior wall, a posterior wall and opposing lateral walls defining at least one graft aperture therebetween;
a block configured to slide inside the graft aperture along a linear path;
a handle for moving the block inside the graft aperture; and
an integrated mechanism for reducing the volume of the graft aperture wherein the handle is used to push the block inward.

2. The spinal fusion implant of claim 1, wherein the handle is removable.

3. A spinal fusion implant, comprising:
an anterior wall, a posterior wall and opposing lateral walls defining a space therebetween;
two flexible interior walls parallel to one another, extending from one side of the space to an opposing side of the space such that a central aperture and two graft apertures are formed;
a block housed in the central aperture; and
an integrated mechanism for reducing the volume of the graft apertures wherein an actuator tool is engaged with the block and the block is rotated from a first position to a second position, thereby flexing the interior walls outward and into the graft apertures.

4. The spinal fusion implant of claim 3, wherein the block is rotated 90° from the first position to the second position.
